(19) United States
(12) Patent Application Publication
(10) Pub. No.: US 2026/0279540 A1
(43) Pub. Date: Sep. 17, 2026

(54) SYSTEM AND METHOD FOR REAL-TIME PREDICTION OF HISTOTRIPSY TREATMENT LOCATION

(71) Applicant: UNIVERSITY-INDUSTRY COOPERATION GROUP OF KYUNG HEE UNIVERSITY, Yongin-si (KR)

(72) Inventors: Ki Joo Pahk, Seoul (KR); SEOKMIN WON, Suwon-si (KR); MUHAMMAD RAFAY MAHMOOD, Cheonan-si (KR); SUNGHYUN KIM, Yongin-si (KR)

(21) Appl. No.: 19/304,642

(22) Filed: Aug. 20, 2025

(30) Foreign Application Priority Data
Mar. 12, 2025 (KR) 10-2025-0032161

(51) Int. Cl. G16H 20/40; G06N 20/00; G16H 30/40; G16H 50/50

(52) U.S. Cl. CPC G16H 20/40; G06N 20/00; G16H 30/40; G16H 50/50

(57) ABSTRACT

Provided is a system and method for real-time prediction histotripsy treatment location, particularly to a system for real-time prediction of histotripsy treatment location, including: a trained AI model configured to predict a focal point position under a specific condition and output a maximum pressure point; and an ultrasound output part configured to output histotripsy ultrasound waves and ultrasound image data including information on subject's skin surface. The trained AI model receives ultrasound image data as input from an input part, and predicts, in real time, a histotripsy focal point that varies according to changes in a skin surface shape and a relative position and distance between an ultrasound transducer and a skin surface, in a processing part.

### STATEMENT REGARDING PRIOR DISCLOSURE UNDER 35 U.S.C. § 102(b)(1)

The subject matter of the present application was disclosed by the inventors prior to the priority date of Mar. 12, 2025. The public disclosure occurred on Sep. 22, 2024. Because the present application was filed within one year of such disclosure, the disclosure qualifies for the exception under 35 U.S.C. § 102(b)(1).

### CROSS-REFERENCE TO RELATED APPLICATIONS

This application claims priority to and the benefit of Korea Patent Application No. 10-2025-0032161 filed in the Korean Intellectual Property Office on Mar. 12, 2025, the entire disclosure of which is incorporated herein by reference.

### BACKGROUND

### Technical Field

The present disclosure relates to a system and method for real-time prediction of histotripsy location.

### Related Art

By using focused ultrasound technology, it is possible to deliver intense acoustic energy, corresponding to tens to hundreds of times atmospheric pressure, to a desired region within the body in a completely non-invasive manner. Furthermore, it enables the selective delivery of thermal or mechanical energy with a spatial resolution of several millimeters (mm).

High Intensity Focused Ultrasound (hereinafter referred to as HIFU), which is based on ultrasound thermal energy, is a technology that concentrates high intensity ultrasound energy on a focal point, generating high temperature heat (above 50° C.), thereby inducing thermal necrosis of tissues. This technology is currently widely used in clinical practice for the treatment of uterine fibroids, adenomyosis, benign prostatic hyperplasia, prostate cancer, metastatic bone tumors, and essential tremor.

Recently, not only the thermal ablation effect but also the physical disruption of biological tissues using the focused ultrasound has been reported in the literature.

The technology known as Histotripsy uses acoustic pressures tens of times greater than those used in conventional HIFU, artificially inducing acoustic cavitation at the ultrasound focal point, and thereby disrupting biological tissues.

Compared to conventional HIFU thermal ablation technology, since the histotripsy offers shorter treatment time and physical disruption of biological tissues, and enables treatment process monitoring through real-time cavitation, it has attracted significant attention as a next-generation promising ultrasound surgical technology that can potentially replace conventional surgical methods applied in clinical practice.

The location of the biological tissue being treated can be verified by monitoring the cavitation generated at the ultrasound focal point using ultrasound imaging techniques (e.g., Ultrasound B-mode). However, there is no method available to provide the practitioner with information on the focal point location in advance of the histotripsy treatment.

### RELATED ART DOCUMENT

### Patent Document

(Patent Document 1) KR Patent No. 10-1625646
(Patent Document 2) KR Patent No. 10-2633843
(Patent Document 3) KR Patent No. 10-1317359

### DETAILED DESCRIPTION

### Technical Problem

Therefore, the present disclosure is contrived to address conventional issues as described above. It is an object to provide a system and method for real-time prediction of histotripsy location, which utilizes an artificial intelligence (hereinafter referred to as AI) model, to predict and display, in real-time, within an ultrasound imaging environment prior to histotripsy treatment, the focal point position that varies depending on changes in the relative position (e.g., distance, angle, etc.) between the focused ultrasound transducer and patient's (or subject's) skin surface.

Further, according to an embodiment of the present disclosure, it is an object to provide a system and method for real-time prediction of histotripsy location, which allows the focal point position to be verified in real-time within an ultrasound imaging environment prior to histotripsy treatment, detects and analyzes, in real time, the patient's (or subject's) skin surface shape as shown in the ultrasound image and the relative position between the focused ultrasound transducer and the skin surface to predict the histotripsy focal point position and display it on the ultrasound image, thereby allowing the user to monitor the histotripsy focal point position in real time prior to histotripsy and preventing side effects that may occur during the actual treatment (if the target treatment location differs from the actual treatment location).

Meanwhile, the technical objects to be achieved by the present disclosure are not limited to those mentioned above, and other technical objects, which are not explicitly stated herein, will be readily understood by those of ordinary skill in the art from the following description.

### Technical Solution

According to a first aspect of the present disclosure, it can be achieved by a system for real-time prediction of histotripsy treatment location, which includes: a trained AI model configured to predict a focal point position under a specific condition and output a maximum pressure point. The trained AI model receives ultrasound image data as input from an input part, and predicts, in real time, a histotripsy focal point that varies according to changes in a skin surface shape and a relative position and distance between an ultrasound transducer and a skin surface, in a processing part.

A second aspect of the present disclosure, it can be achieved by a training method of trained AI model of the system for real-time prediction of histotripsy treatment location according the aforementioned first aspect, which includes steps of: inputting, into an input part, input data including focused ultrasound transducer information, various surface shapes of a biological skin phantom, a surface angle of the phantom, and a distance between a focused ultrasound transducer and a phantom surface; receiving the input data as input by a numerical analysis part and simulating an acoustic pressure distribution to obtain a maximum pressure point and a focal point of a focused ultrasound; and receiving the simulation data as input and training an AI model, wherein the trained model predicts a focal point position under a specific condition and outputs a maximum pressure point.

In the second aspect of the present disclosure, it is characterized in that an output of the numerical analysis part for the input data represents an ultrasound acoustic pressure and includes an output of the maximum pressure point, being utilized as a label for the AI model.

Further, the second aspect of the present disclosure, it is characterized in that the training method further includes a step of: performing image processing to represent a phantom layer as an image, based on the input data, wherein the image obtained through the image processing includes various surface shapes of the phantom, a surface angle of the phantom, and information on a relative position and distance between the focused ultrasound transducer and the phantom surface, and is applied to the AI model.

In the first aspect of the present disclosure, it is characterized in that the system further includes a preprocessing part configured to preprocess data collected from the input part for compatibility with the AI model input, wherein the preprocessing process by the preprocessing part includes noise removal, image resizing, and region of interest extraction.

Further, in the first aspect of the present disclosure, it is characterized in that the preprocessing process includes steps of: extracting consecutive frames in a temporal sequence from ultrasound B-mode image data; converting each frame into independent image data; and performing a filtering process to remove unnecessary external or internal noises occurring in the ultrasound image; converting a resolution and size of the framed images to conform with an input specification required by the trained AI model; and normalizing a value range of the input data.

Yet further, in the first aspect of the present disclosure, it is characterized in that the AI model receives preprocessed ultrasound images as input and applies an AI-based real-time prediction algorithm for a histotripsy focal point.

Yet further, in the first aspect of the present disclosure, it is characterized in that the prediction algorithm analyzes, in real time, morphological structures and characteristics near a surface of a human body from the input B-mode image data, and extracts key data for focal point prediction therefrom.

Yet further, in the first aspect of the present disclosure, it is characterized in that the prediction algorithm is a deep learning-based network algorithm trained to predict a maximum pressure point of histotripsy according to surface shape, and a relative position and distance between a transducer and the surface, wherein the algorithm predicts a focal point based on data of various surface shapes accumulated during a training process.

Yet further, in the first aspect of the present disclosure, it is characterized in that the system further includes a display part configured to visually indicate, on the ultrasound B-mode, in real time, the histotripsy focal point predicted by the AI model.

A third aspect of the present disclosure, it can be achieved by a method for real-time prediction of histotripsy treatment location, including steps of: forming a trained AI model according to the training method in accordance with the aforementioned second aspect; and receiving ultrasound image data as input from an input part by the trained AI model, and predicting, in real time, a histotripsy focal point that varies according to changes in a skin surface shape and a relative position and distance between a transducer and a skin surface, in a processing part.

In the third step of the present disclosure, it is characterized in that, after the output step, the method further includes a step of comprising step of performing preprocessing the ultrasound image data for compatibility with the AI model by a preprocessing part, wherein the preprocessing process by the preprocessing part includes noise removal, image resizing, and region of interest extraction.

Further, in the third step of the present disclosure, it is characterized in that the preprocessing performing step includes steps of: extracting consecutive frames in a temporal sequence from ultrasound B-mode image data; converting each frame into independent image data; and performing a filtering process to remove unnecessary external or internal noises occurring in the ultrasound image; converting a resolution and size of the framed images to conform with an input specification required by the trained AI model; and normalizing a value range of the input data.

Yet further, in the third step of the present disclosure, it is characterized in that the AI model receives preprocessed ultrasound images as input and applies an AI-based real-time prediction algorithm for a histotripsy focal point, wherein the prediction algorithm analyzes, in real time, morphological structures and characteristics near a surface of a human body from the input B-mode image data, and extracts key data for focal point prediction therefrom.

Yet further, in the third step of the present disclosure, it is characterized in that the prediction algorithm is a deep learning-based network algorithm trained to predict a maximum pressure point of histotripsy according to surface shape, and a relative position and distance between a transducer and the surface, wherein the algorithm predicts a focal point based on data of various surface shapes accumulated during a training process.

Yet further, in the third step of the present disclosure, it is characterized in that the method includes a step of visually indicating, on the ultrasound B-mode, in real time, the histotripsy focal point predicted by the AI model, by a display part.

A fourth aspect of the present disclosure, it can be achieved by a program for real-time prediction of histotripsy treatment location, which is readable and executed by a computer. It is characterized in that the program is configured to execute the prediction method according to the aforementioned third aspect of the present.

### Advantageous Effects

According to a system and method for real-time prediction of histotripsy location in accordance of the embodiment of the present disclosure, there are effects of predicting and displaying, in real-time, within an ultrasound imaging environment prior to histotripsy treatment, the focal point position that varies depending on changes in the relative position (e.g., distance, angle, etc.) between the focused ultrasound transducer and patient's (or subject's) skin surface, by utilizing an AI model.

Additionally, according to a system and method for real-time prediction of histotripsy location in accordance of the embodiment of the present disclosure, there are effects of allowing the focal point position to be verified in real-time within an ultrasound imaging environment prior to histotripsy treatment, detecting and analyzing, in real time, the patient's (or subject's) skin surface shape as shown in the ultrasound image and the relative position between the focused ultrasound transducer and the skin surface to predict the histotripsy focal point position and display it on the ultrasound image, thereby allowing the user to monitor the histotripsy focal point position in real time prior to histotripsy and preventing side effects that may occur during the actual treatment (if the target treatment location differs from the actual treatment location).

Meanwhile, advantageous effects to be obtained from the present disclosure are not limited to those mentioned above, and other effects, which are not explicitly stated herein, will be readily understood by those of ordinary skill in the art from the following description.

### BRIEF DESCRIPTION OF THE DRAWINGS

The accompanying drawings of this specification exemplify a preferred embodiment of the present disclosure, the spirit of the present disclosure will be more clearly understood from the following detailed description taken in conjunction with the accompanying drawings, and thus it will be understood that the present disclosure is not limited to only contents illustrated in the accompanying drawings;

FIG. 1 is a block diagram showing a training process of an ultrasound image-based histotripsy focal point prediction AI model according to an embodiment of the present disclosure,

FIG. 2 is a flowchart showing a training process of an ultrasound-based histotripsy focal point prediction AI model according to an embodiment of the present disclosure,

FIG. 3 is a block diagram showing a system for real-time prediction of a histotripsy treatment location according to an embodiment of the present disclosure,

FIG. 4 is a flowchart showing a method for real-time prediction of a histotripsy treatment location according to an embodiment of the present disclosure,

FIG. 5A is a photograph of an experimental setup used for performing histotripsy according to an experimental example of the present disclosure,

FIG. 5B shows ultrasound B-mode image data during exposure according to an experimental example of the present disclosure,

FIG. 5C is an AI model input image according to an experimental example of the present disclosure,

FIG. 5D shows a comparison of focal point positions in the experiment, the AI model and the simulation according to an experimental example of the present disclosure,

FIG. 5E is an enlarged view of three focal points in FIG. 5D, and

FIG. 5F is a histotripsy focal point error table between AI model predictions and experimental results based on surface shape according to an experimental example of the present disclosure.

### DETAILED DESCRIPTION

Hereinafter, the aforementioned aims, other aims, features and advantageous effects of the present disclosure will be understood easily referring to preferable embodiments related to the accompanying drawings. However, the present disclosure is not limited to embodiments described in this specification, and may be embodied into other forms. Preferably, the embodiments in this specification are provided in order to allow disclosed contents to be exhaustive and to communicate the concept of the present disclosure to those skilled in the art.

In this specification, when a certain element is placed on another element, this means that it may be formed directly thereon or that the third element may be interposed between them. Further, in the drawings, the thickness of an element may be overstated in order to explain the technical content thereof efficiently.

The embodiments described in this specification will be explained with reference to a cross-sectional view and/or a plane view. In the drawings, the thickness of a film and a region may be overstated in order to explain the technical content thereof efficiently. Accordingly, the form of exemplary drawings for a fabrication method and/or an allowable error et cetera may be reformed. Thus, the embodiments according to the present disclosure are not limited to specific forms illustrated herein, but may include variations in the form resulting from the fabrication method. For example, the region illustrated with perpendicular lines may have a form to be rounded or with a predetermined curvature. Thus, regions exemplified in the drawings have attributes, and shapes thereof exemplify specific forms rather than limiting the scope of the present disclosure. In the various embodiments of this specification, terms such as ‘first’ and ‘second’ et cetera are used to describe various elements, but these elements should not be limited to such terms. These terms are merely used to distinguish one element from others. The embodiments explained and exemplified herein may include complementary embodiments thereto.

The terms used in this specification are to explain the embodiments rather than limiting the present disclosure. In this specification, the singular expression includes the plural expression unless specifically stated otherwise. The terms, such as ‘comprise” and/or “comprising” do not preclude the potential existences of one or more elements.

When describing the following specific embodiments, various kinds of specific contents are made up to explain the present disclosure in detail and to help understanding thereof. However, it will be apparent for those who have knowledge to the extent of understanding the present disclosure that the present disclosure can be used without any of these specific contents. In a certain case when describing the present disclosure, the content that is commonly known to the public but is largely irrelevant to the present disclosure is not described in order to avoid confusion.

Hereinafter, the configuration, functions and operating method of a system and method for real-time prediction of histotripsy treatment location according to an embodiment of the present will be described.

Histotripsy is a technology that uses shock waves with high acoustic pressure and artificially induces acoustic cavitation at the ultrasound focal point, thereby physically disrupting biological tissues without thermal damage.

A system for real-time prediction of histotripsy treatment location according to the present disclosure, is configured to include a trained AI model that predicts a focal point position under a specific condition and outputs a maximum pressure point.

The ultrasound output part outputs histotripsy ultrasound waves and ultrasound image data including information on subject's skin surface.

Accordingly, the system for real-time prediction of histotripsy treatment location according to the present disclosure receives the ultrasound image data as input

Accordingly, in the real-time prediction system for a histotripsy treatment location according to an embodiment of the present disclosure, the trained AI model receives the ultrasound image data as input from an input unit and the histotripsy focal point that changes depending on the skin surface morphology and/or relative position and distance between the skin surface and the ultrasound transducer is predicted in real time, in a processing unit.

Hereinafter, a training process of an ultrasound image-based histotripsy focal point prediction AI model according to an embodiment of the present disclosure will be described.

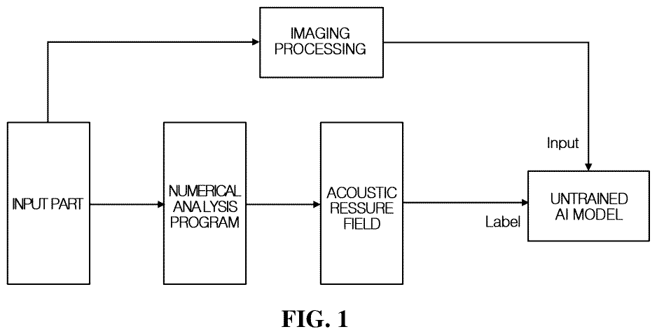

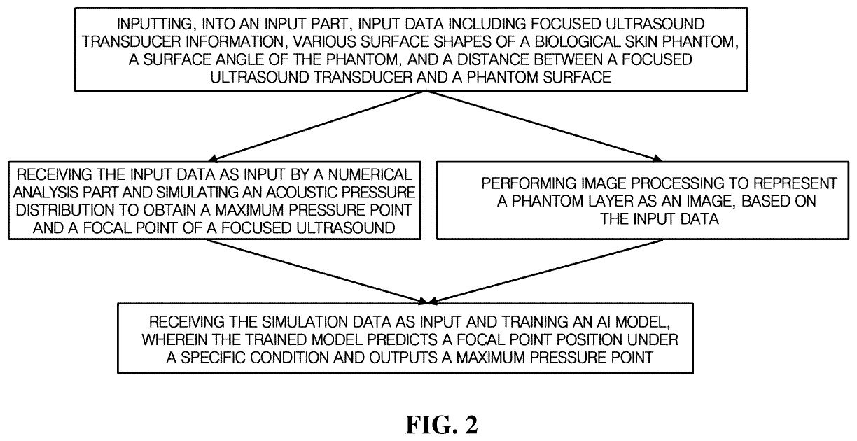

FIG. 1 is a block diagram showing a training process of an ultrasound image-based histotripsy focal point prediction AI model according to an embodiment of the present disclosure. FIG. 2 is a flowchart showing a training process of an ultrasound-based histotripsy focal point prediction AI model according to an embodiment of the present disclosure.

A training method of an AI model for real-time prediction of histotripsy treatment location may include steps of: inputting, into an input part, input data including focused ultrasound transducer information, various surface shapes of a biological skin phantom, a surface angle of the phantom, and a distance between a focused ultrasound transducer and a phantom surface; receiving the input data as input by a numerical analysis part and simulating an acoustic pressure distribution to obtain a maximum pressure point and a focal point of a focused ultrasound; and receiving the simulation data as input and training an AI model, wherein the trained model predicts a focal point position under a specific condition and outputs a maximum pressure point.

Additionally, an output of the numerical analysis part for the input data represents an ultrasound acoustic pressure and includes an output of the maximum pressure point, thereby being utilized as a label for the AI model. The training method further includes a step of: performing image processing to represent a phantom layer as an image, based on the input data, wherein the image obtained through the image processing includes various surface shapes of the phantom, a surface angle of the phantom, and information on a relative position and distance between the focused ultrasound transducer and the phantom surface, thereby allowing the image to be applied to the AI model.

In a specific embodiment of the present disclosure, the AI model may be trained as follows using a tissue phantom having physical properties or acoustic state variables similar to those of biological tissues.

Input (parameters) need to be provided to a numerical analysis program that simulates an acoustic pressure distribution to obtain the maximum pressure point (focal point) of the focused ultrasound. The input may include focused ultrasound transducer information (diameter, radius of curvature, frequency), various surface shapes of the tissue phantom, a surface angle of the tissue phantom, and a distance between the focused ultrasound transducer and the tissue phantom surface. The output of the numerical analysis program for the input represents the ultrasound acoustic pressure and includes the output of the maximum pressure point, thereby being utilized as a label for the AI model.

Image processing is performed to represent the tissue phantom layer as an image based on the information used as input to the numerical analysis program. The obtained image therethrough includes various surface shapes of the tissue phantom, a surface angle of the tissue phantom, information on the relative position and distance between the focused ultrasound transducer and the phantom surface, thereby allowing the image to be applied as input of the AI model.

The AI model is trained using the label of the maximum pressure point of the ultrasound focal point and the image including various information, as input. The trained model predicts the focal point under a specific condition and outputs the maximum pressure point.

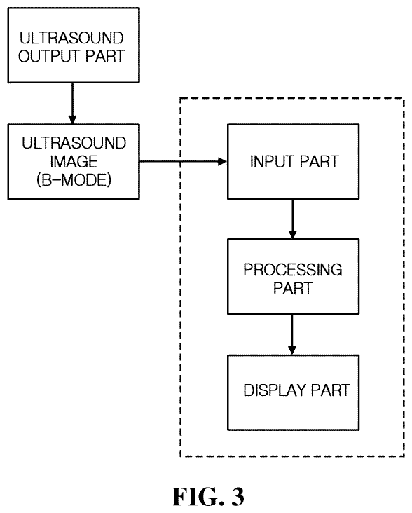

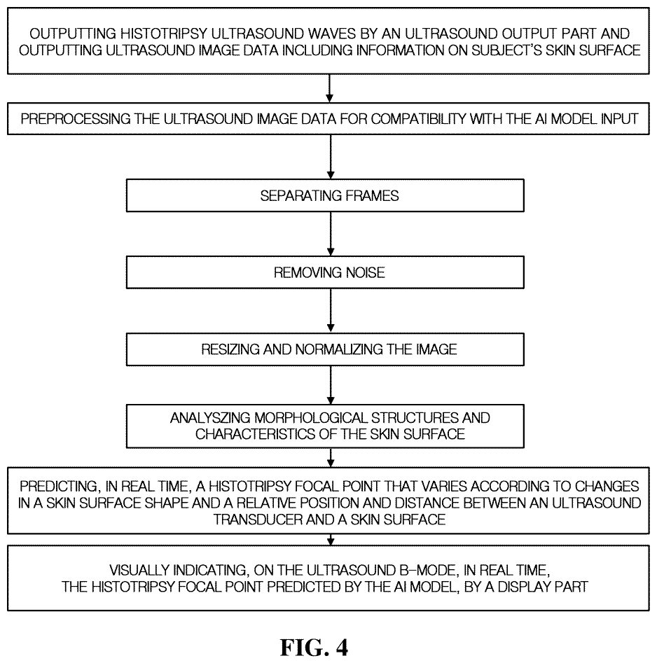

FIG. 3 is a block diagram showing a system for real-time prediction of a histotripsy treatment location according to an embodiment of the present disclosure. FIG. 4 is a flowchart showing a method for real-time prediction of a histotripsy treatment location according to an embodiment of the present disclosure.

The system for real-time prediction of histotripsy treatment location according to an embodiment of the present disclosure includes an image preprocessing module that acquires an ultrasound image and converts the acquired image into an input of the trained AI model, and an AI model that analyzes the preprocessed data to predict the histotripsy focal in real time.

As shown in FIG. 3, AI-based histotripsy focal point prediction system includes: an input part configured to receive ultrasound image data including skin surface information of a patient (or subject) for predicting the focal point; an ultrasound output part configured to output histotripsy ultrasound waves; a processing part configured to perform real-time prediction of the histotripsy focal point, which varies according to changes in a skin surface shape and a relative position and distance between an ultrasound transducer and a skin surface, using the AI model, by the ultrasound waves generated from the output part; and a display part configured to indicate the predicted focal point information in real time.

In addition, the system for real-time prediction of histotripsy treatment location may further include a preprocessing part configured to preprocess the data collected from the input part for compatibility with the AI model input. The preprocessing process by the preprocessing part may include noise removal, image resizing, and region of interest extraction.

Particularly, the preprocessing process may include steps of: extracting consecutive frames in a temporal sequence from ultrasound B-mode image data; converting each frame into independent image data; and performing a filtering process to remove unnecessary external or internal noises occurring in the ultrasound image; converting a resolution and size of the framed image to conform with an input specification required by the trained AI model; and normalizing a value range of the input data.

The preprocessing process is an important step that enables the AI model to more precisely predict histotripsy focal point.

In the preprocessing process, the frame separation step is a step of extracting consecutive frames in a temporal sequence from the ultrasound B-mode image data and converting each frame into independent image data.

The noise removing step includes the filtering process for removing unnecessary external or internal noises occurring in the ultrasound image, thereby corresponding to a step for improving image quality and increasing analysis accuracy.

Additionally, the image resizing and normalizing step is a process for converting the resolution and size of the framed images to conform with an input specification required by the trained AI model and normalizing the value range of the input data.

In the system for real-time prediction of histotripsy treatment location, the AI model receives preprocessed ultrasound images as input and applies an AI-based real-time prediction algorithm for a histotripsy focal point.

The prediction algorithm analyzes, in real time, morphological structures and characteristics near a surface of a human body from the input B-mode image data, and extracts key data for focal point prediction therefrom.

The prediction algorithm is a deep learning-based network algorithm trained to predict a maximum pressure point of histotripsy according to surface shape, and a relative position and distance between a transducer and the surface, and wherein the algorithm predicts a focal point based on data of various surface shapes accumulated during a training process.

In other words, the AI model receives the preprocessed ultrasound image as input and includes the AI-based real-time prediction algorithm for performing the following functions:
(a) Analysis of morphological structures and characteristics of the skin surface: a function of analyzing, in real time, morphological structures and characteristics near a surface of a human body from the input B-mode image data, and extracting key data for focal point prediction therefrom; and
(a) Focal point prediction algorithm: a deep learning-based network algorithm trained to predict a maximum pressure point of histotripsy according to surface shape, and a relative position and distance between a transducer and the surface, wherein the algorithm predicts a focal point based on data of various surface shapes accumulated during a training process.

Furthermore, the system for real-time prediction of histotripsy treatment location may include a display part configured to visually indicate, on the ultrasound B-mode, in real time, the histotripsy focal point predicted by the AI model.

The display part visually indicates, on the ultrasound B-mode, in real time, the histotripsy focal point predicted by the AI model, thereby allowing the user to immediately response to the histotripsy focal point that varies according to the shape and position of patient's skin surface, thereby contributing to improved accuracy and efficiency of histotripsy treatment.

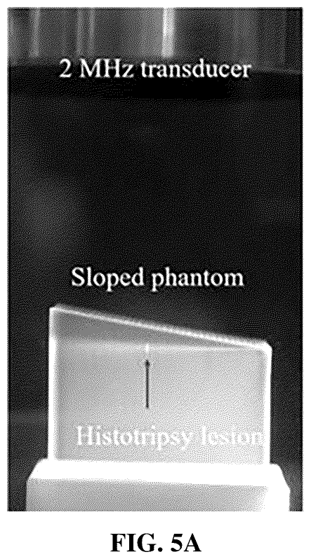

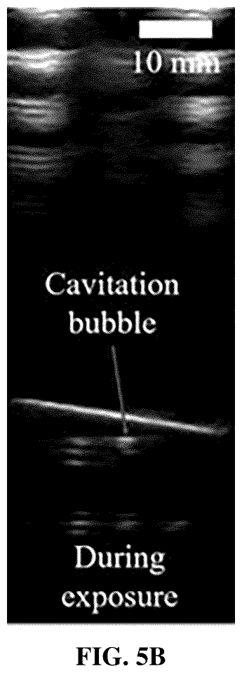

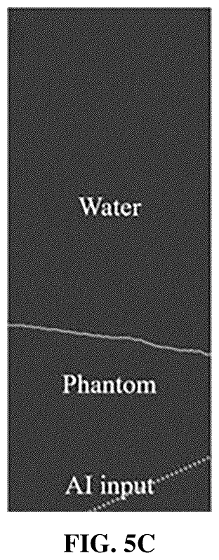

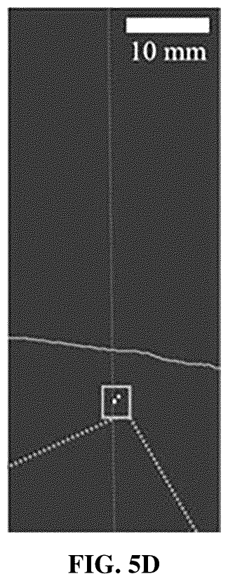

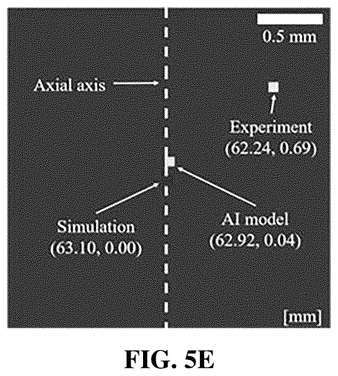

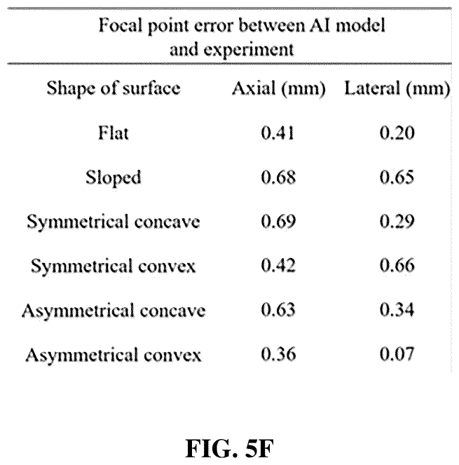

FIG. 5A is a photograph of an experimental setup used for performing histotripsy according to an experimental example of the present disclosure. FIG. 5B shows ultrasound B-mode image data during exposure according to an experimental example of the present disclosure. FIG. 5C is an AI model input image according to an experimental example of the present disclosure. FIG. 5D shows a comparison of focal point positions in the experiment, the AI model and the simulation according to an experimental example of the present disclosure. FIG. 5E is an enlarged view of three focal points in FIG. 5D. FIG. 5F is a histotripsy focal point error table between AI model predictions and experimental results based on surface shape according to an experimental example of the present disclosure.

The histotripsy focal point position predicted on the AI model was compared to the focal point position measured in experiments and the simulated focal point position through the numerical analysis program (simulation).

As a result, it was confirmed that the error between the focal point position predicted in the AI model and the focal point position measured in actual experiments was within 1 mm, indicating a very small error.

Further, the configuration and method of the embodiments as described above are not restrictively applied to the aforementioned apparatus and method. The whole or part of the respective embodiments may be selectively combined so as to make various modifications of the embodiments.

1. A system for real-time prediction of histotripsy treatment location, comprising:
a trained AI model configured to predict a focal point position under a specific condition and output a maximum pressure point, wherein
the trained AI model receives ultrasound image data as input from an input part, and predicts, in real time, a histotripsy focal point that varies according to changes in a skin surface shape and/or a relative position and distance between an ultrasound transducer and a skin surface, in a processing part.

2. A training method of trained AI model of the system for real-time prediction of histotripsy treatment location according to claim 1, comprising steps of:
inputting, into an input part, input data including focused ultrasound transducer information, various surface shapes of a biological skin phantom, a surface angle of the phantom, and a distance between a focused ultrasound transducer and a phantom surface;
receiving the input data as input by a numerical analysis part and simulating an acoustic pressure distribution to obtain a maximum pressure point and a focal point of a focused ultrasound; and
receiving the simulation data as input and training an AI model, wherein the trained model predicts a focal point position under a specific condition and outputs a maximum pressure point.

3. The training method of trained AI model of the system for real-time prediction of histotripsy treatment location of claim 2, wherein
an output of the numerical analysis part for the input data represents an ultrasound acoustic pressure and includes an output of the maximum pressure point, being utilized as a label for the AI model.

4. The training method of trained AI model of the system for real-time prediction of histotripsy treatment location of claim 3, further comprising a step of:
performing image processing to represent a phantom layer as an image, based on the input data, wherein
the image obtained through the image processing includes various surface shapes of the phantom, a surface angle of the phantom, and information on a relative position and distance between the focused ultrasound transducer and the phantom surface, and is applied to the AI model.

5. The system for real-time prediction of histotripsy treatment location of claim 1, further comprising:
a preprocessing part configured to preprocess data collected from the input part for compatibility with the AI model input, wherein
the preprocessing process by the preprocessing part includes noise removal, image resizing, and/or region of interest extraction.

6. The system for real-time prediction of histotripsy treatment location of claim 5, wherein
the preprocessing process comprises steps of:
extracting consecutive frames in a temporal sequence from ultrasound B-mode image data;
converting each frame into independent image data; and/or
performing a filtering process to remove unnecessary external or internal noises occurring in the ultrasound image;
converting a resolution and size of the framed images to conform with an input specification required by the trained AI model; and/or
normalizing a value range of the input data.

7. The system for real-time prediction of histotripsy treatment location of claim 6, wherein
the AI model receives preprocessed ultrasound images as input and applies an AI-based real-time prediction algorithm for a histotripsy focal point.

8. The system for real-time prediction of histotripsy treatment location of claim 7, wherein
the prediction algorithm analyzes, in real time, morphological structures and characteristics near a surface of a human body from the input B-mode image data, and extracts key data for focal point prediction therefrom.

9. The system for real-time prediction of histotripsy treatment location of claim 7, wherein
the prediction algorithm is a deep learning-based network algorithm trained to predict a maximum pressure point of histotripsy according to surface shape, and a relative position and distance between a transducer and the surface, and wherein
the algorithm predicts a focal point based on data of various surface shapes accumulated during a training process.

10. The system for real-time prediction of histotripsy treatment location of claim 8, further comprising:
a display part configured to visually indicate, on the ultrasound B-mode, in real time, the histotripsy focal point predicted by the AI model.

11. A method for real-time prediction of histotripsy treatment location, comprising steps of:
forming a trained AI model according to the training method of claim 2; and
receiving ultrasound image data as input from an input part by the trained AI model, and predicting, in real time, a histotripsy focal point that varies according to changes in a skin surface shape and/or a relative position and distance between a transducer and a skin surface, in a processing part.

12. The method for real-time prediction of histotripsy treatment location of claim 11, further comprising a step of:
performing preprocessing the ultrasound image data for compatibility with the AI model by a preprocessing part, wherein
the preprocessing process by the preprocessing part includes noise removal, image resizing, and/or region of interest extraction.

13. The method for real-time prediction of histotripsy treatment location of claim 12, wherein
the preprocessing performing step comprises steps of:
extracting consecutive frames in a temporal sequence from ultrasound B-mode image data;
converting each frame into independent image data; and/or
performing a filtering process to remove unnecessary external or internal noises occurring in the ultrasound image;
converting a resolution and size of the framed image to conform with an input specification required by the trained AI model; and/or
normalizing a value range of the input data.

14. The method for real-time prediction of histotripsy treatment location of claim 13, wherein
the AI model receives preprocessed ultrasound images as input and applies an AI-based real-time prediction algorithm for a histotripsy focal point; and
the prediction algorithm analyzes, in real time, morphological structures and characteristics near a surface of a human body from the input B-mode image data, and extracts key data for focal point prediction therefrom.

15. The method for real-time prediction of histotripsy treatment location of claim 14, wherein
the prediction algorithm is a deep learning-based network algorithm trained to predict a maximum pressure point of histotripsy according to surface shape, and a relative position and distance between a transducer and the surface, and wherein
the algorithm predicts a focal point based on data of various surface shapes accumulated during a training process.

16. The method for real-time prediction of histotripsy treatment location of claim 15, further comprising a step of:
visually indicating, on the ultrasound B-mode, in real time, the histotripsy focal point predicted by the AI model, by a display part.

17. A program for real-time prediction of histotripsy treatment location, which is readable and executed by a computer, wherein
the program is configured to execute the prediction method according to claim 11.

18. A method for real-time prediction of histotripsy treatment location, comprising steps of:
forming a trained AI model according to the training method of claim 3; and
receiving ultrasound image data as input from an input part by the trained AI model, and predicting, in real time, a histotripsy focal point that varies according to changes in a skin surface shape and/or a relative position and distance between a transducer and a skin surface, in a processing part.

19. A method for real-time prediction of histotripsy treatment location, comprising steps of:
forming a trained AI model according to the training method of claim 4; and
receiving ultrasound image data as input from an input part by the trained AI model, and predicting, in real time, a histotripsy focal point that varies according to changes in a skin surface shape and/or a relative position and distance between a transducer and a skin surface, in a processing part.
